(19) United States
(12) Patent Application Publication
(10) Pub. No.: US 2017/0042912 A1
(43) Pub. Date: Feb. 16, 2017

(54) PHARMACEUTICAL COMPOSITION FOR TREATING CANCER IN AN INDIVIDUAL SUBJECT SUFFERING CANCER

(71) Applicant: MA SHEN KAI RUEI CO., LTD., Tainan City (TW)

(72) Inventor: PO-LIANG CHEN, Taipei City (TW)

(21) Appl. No.: 14/852,712

(22) Filed: Sep. 14, 2015

(30) Foreign Application Priority Data
Aug. 12, 2015 (TW) 104126303

(51) Int. Cl. A61K 31/575; A61K 31/4015; A61K 31/122

(52) U.S. Cl. CPC A61K 31/575; A61K 31/122; A61K 31/4015

(57) ABSTRACT

A pharmaceutical composition for treating cancer in an individual, comprising at least a pharmaceutically effective amount of a novel target HCC reversal signal substance, and a pharmaceutically acceptable ingredient comprising a vehicle, a carrier, a diluent or an excipient; in which the ratio among each component of the pharmaceutical composition may be adjustable depending on the type of cancer; wherein the novel target HCC reversal signal substance comprises at least one selected from the group consisting of antroquinonol (AQL), antrocinnamonin A (ACA), antroquinonol B (AQB), antroquinonol D (AQD), dehydroeburicoic acid (DEA), dehydrosulphurenic acid (DSA), zhankuic acid A (ZAA), zhankuic acid C (ZAC), antcin K (ANK), antcin C (ANC), and a mixture thereof.

### BACKGROUND OF THE INVENTION

1. Field of the Invention

The present invention relates to a pharmaceutical composition for treating cancer in an individual subject suffering cancer in need such a treatment; particularly relates to a pharmaceutical composition for treating cancer in an individual subject suffering cancer in need such a treatment, comprising at least a pharmaceutically effective amount of a novel target HCC reversal signal substance, and a pharmaceutically acceptable vehicle, a carrier, a diluent or an excipient; in which the ratio among each component of the pharmaceutical composition may be adjustable depending on the type of cancer.

2. Descriptions of Related Art

In Taiwan, cancer or malignancy has become one of the top ten causes of death of people in recent decades. The term “tumor” in medical science, includes benign and malignant tumors, usually refers to abnormal cell proliferation forming cumbersome, even violations of surrounding or distant tissue causing effects to its normal physiological function. Common cancers includes liver cancer, colorectal cancer, colorectal cancer, esophageal cancer, stomach cancer, leukemia, lymphoma, nasopharyngeal cancer, brain tumors, lung cancer, breast cancer, cervical cancer, leukemia and bone cancer, and others. Among them, the lung cancer, stomach cancer and liver cancer respectively accounting for 18% 10% and 9% of deaths of people, are known as the top three lethal cancers.

A variety of malignant tumors is generally referred to as “cancer”. The growth and division of cancer cells are generally out of control and the cancer cells can invade normal tissue and surrounding tissue via the body circulatory system, the lymphatic system or be transferred to the chamber away from its site of origin of the growth. It thus serious challenges to human health and safety if he or she gets cancer. The treatment for treating cancer can be generally divided into surgery, chemotherapy, radiation therapy, targeted therapy, immunotherapy, hormone therapy, cryotherapy, heating treatment, angiogenesis inhibitor therapy, Chinese medicine, and Chinese herbal medicine treatment.

The surgery, radiotherapy and chemotherapy in a variety of cancer treatments, are generally used as the primary means of clinical treatment, and in which different treatments may be selected based on the type, location and time of cancer in a patient. Surgical resection mainly involves a removal means of removing a tissue and/or a cell on a cancer site in a subject. Radiotherapy and chemotherapy may damage normal cells of the human body and also raise side effects while killing the tumor cells in patients, as well as these radiotherapy and chemotherapy may further cause worse quality of life, poor survival rate and others to patients. However, whether surgical removal, chemical treatment, or radiation treatment is taken as a therapy means for treating cancer, each of them involves destruction of human cells, tissues and even organs and pertain to irreversible methods to a patient.

As to the results of study of the phenomenon of cell death, the molecular mechanism and other research, people has a novel and more understanding in those drugs for treating cancer and comes to have sufficient knowledge to know how to screen out more effective drugs for treating cancer. As to a method causing less secondary irreversible damage to cancer patients, for example, the means of using pharmaceutical preparations comprising herbal or containing components of herbal extracts to carry out the treatment for treating specific signaling pathways of tumor cells or cancer-specific target sites, are getting more and more concern by people greatly. In these drugs or pharmaceutical composition for inducing apoptosis or death of cancer cells, for example, addition of an additive ingredient comprising Antrodia camphorata extracts as pharmaceutically active substance is generally used and also become a rapid increase trend in recent years.

Antrodia camphorata is a perennial mushrooms having medical efficiency and can be taken as curative food. A fruiting body of Antrodia camphorata is generally a sessile and a corky to woody type, with a strong camphor arom, having a various configurations such as plate-like, bell-shaped, horseshoe-shaped, or tower-shaped profiles, and often grows tightly affixed to the wood surface. The fruiting bodies of Antrodia camphorate has primary seasonal red color in its surface, which becomes milky white, reddish brown, light brown or light brown with growth gradually; and often has a radial periphery of around semicircular or irregular shape in expand growth.

Fruiting body of Antrodia camphorate grows in heartwood wall surrounding curved inwards of cattle camphor (Cinnamoum kanehirai Hay), one protected species in Taiwan, and it belongs to the wood decay fungi capable of decomposing cellulose timber and causing wood rot. The wild Antrodia camphorate generally grows in an environment which is dark, damp and low temperature, in a very slow growth rate among the altitude, and especially, and it is thus required to take a long time for growth of fruiting body of wild Antrodia camphorates. Since the number and distribution of are both extremely rare, and also for the sake of problems such as artificial illegal logging, the cattle camphor tree belongs to a rare species required to be protected. Further, the wild Antrodia camphorates is famous and especially, it has been called as “forest ruby” in Taiwan, because of not only very expensive but also rare in number and amount.

Antrodia (Antrodia camphorata, Taiwanofungus camphorates), also known as Zhang raw thin volvatus, stout camphor mushroom, Zhang Chi, Zhang Zhi red blood fungus, mushroom inside camphor, or red camphor mushroom, which belong to the Kingdom Fungi (Fungi) Basidiomycota, Basidiomycotina, Homobasidiomycetes, Aphyllophorales, Polyporaceae, Antrodia, a unique Taiwan fungus in taxonomy. According to results of phytochemistry studies, the fruiting bodies of Antrodia camphorates contain polysaccharide (30-50%), triterpenoids (30%), steroids, superoxide dismutase and amino acids.

In medicine, the active ingredients such as triterpenoids, polysaccharides, nucleic acids, polypeptides, phytosterols, etc. which are separated from Antrodia camphorates, have been confirmed that they have specific pharmaceutical effects of showing active effectiveness in inhibitions of cholesterol synthesis, anticancer, antihypertensive and others. For example, if a fine powder of the fruiting bodies of Antrodia camphorates obtained by finely grounding after drying or boiled extract is orally administered into a subject, it shows pharmaceutical activities such as especially in detoxification, anti-inflammatory, anti-tumor, inhibition of angiogenesis and treatment capability of liver disease. Briefly, because of having medical effects, Antrodia camphorates can be used as not only nutritional supplements, but also can be regarded as medicinal herbs, anticancer active ingredients or agents and often used in improvement of health.

It was confirmed extracts of Antrodia camphorates have significant performance in medical efficacy, for example high anti-inflammatory activities such as in inhibiting TNF-α hormone production, and reducing phosphorylation of IκB and avoiding NF-κB into the nucleus, thereby inhibiting the expressions of iNOS and COX-2 protein presented at further downstream, as well as decreasing the generation of NO- and PGE-2 and the likes. For example, to mouse macrophages (EOC13.31 cells), the extracts of Antrodia camphorates were shown high capability in inhibiting the expressions of TNF-α, iNOS and COX-2, and then effectively showing anti-inflammatory effects together with reducing LPS-induced inflammation in the mice body. In extracts of Antrodia camphorates, the experiments confirmed Antroquinonol B (AQB), 4-acetyl-Antroquinonol B (4-acetyl-AQB), 2,3-(methylenedioxy)-6-methyl-benzene-1,4-diol, and 2,4-dimethoxy-6-methyl-benzene-1,3-diol and others are anti-inflammatory active ingredient capable of effectively reducing the amount of free radicals of nitric oxide, which may be generated by LPS-induction in murine macrophages RAW264.7.

In addition to the efficacy of anti-inflammatory, a lot of research has been confirmed that Antrodia camphorates shows high performance in anti-cancer performance. For example, in medical science, it has been proven that by using active ingredients of Antrodia camphorates in treatment of a variety of cancer cells, can effectively activate the mitochondrial Bax, suppress anti-Bcl-2 and Bcl-xL proteins in junction therewith, as well as can increase the permeability of outer membrane of mitochondria such that calcium easily access into the inside of mitochondria, release Cytochrome C to bond with Apaf-1, and activate Caspase-9 and Caspase-3 at downstream, and then effectively induce apoptosis in all kinds of cancer cells.

It is also known that extracts of Antrodia camphorates have other medical effects. For example, it can effectively apoptosis (cell death) of liver cancer cells (HepG2), inhibit activity of calpains in liver cancer cells (Hep-3B) and then induce apoptosis in Hep-3B cells, as well as can significantly inhibit PLC/PRF/5 HCC cell growth and increase the expressions of TIMP-1 and TIMP-2. Besides, it is confirmed that the fruiting body powder of Antrodia camphorates can significantly reduce the formation of cholesterol and fat in the liver of rats, and can inhibit expressions of HMG Co-A, SREBT-1c, acetyl-CoA carboxylase, FAS and Malic enzyme gene.

Further, it is reported if a concentration of 50 μg/ml is administered, the extracts of Antrodia camphorates can inhibit the growth of human bladder cancer (T24 cells) and the expression of Cdc2 and CyclinB1 protein; can induce the phosphorylation of ERK and JNK (P-38 did not show) in human oral cell (OC2) and then induce apoptosis in the OC2 cell to. According to the experimental results, it shows that in the extracts of Antrodia camphorates, the compound of Zhankuic acid A active ingredients have capability of inducing apoptosis in human HL60 leukemia cells.

Furthermore, it is also reported that in the extracts of Antrodia camphorates, can inhibit the survival ratio of human breast cancer cells (MCF-7 cells), inhibit the phosphorylation of Akt, induce activation of Bax and Caspase-3 and apoptosis in cells.

In addition, some studies have confirmed that the ethanol extracts of Antrodia camphorates can make the cell cycle of human lung cancer cells (H441GL cells) remain in the G0/G1 phase, and can induce the activation of Caspase-9 and Caspase-3 signal path and further induce apoptosis in cells.

In addition, it is reported that the triterpenoids of Antrodia camphorates showed strongest insecticidal activity (IC50=22.3˜70.5 μg/ml) to human colon cancer cells (HT-29 cells, HCT116 cells, and SW480 cell). In addition, it was confirmed that in case of using 4,7-dimethoxy-5-methyl-1,3-benzo-oxadiazole (SY-1) presented in extracts of Antrodia camphorates to treat a cancer cell, if lower doses of SY-1 is used, it can make the cell cycle of human colorectal cancer cells (COLO 205) remain in the G0/G1 phase, while if higher doses of SY-1 is used, it can induce programmed death of cell, and SY-1 does not have an impact on the normal intestinal epithelial cells (FHC cells).

In addition to anti-inflammatory, anti-cancer effectiveness, it also confirmed that polysaccharide component in the extracts of Antrodia camphorates have excellent efficacy for the treatment of asthma, for example, it can improve the production amount of IL-10 and IL-12 in mice, and also can inhibit the proliferation of CD4+T cells and differentiation of Th2 cells. Besides, it also confirmed that polysaccharides isolated from Antrodia camphorates can inhibit the expression of cyclin D1 (cyclin D1) through inhibiting signal transduction of vascular endothelial growth factor receptor (VEGF receptor) and it thus can inhibit angiogenesis.

Although the extracts of Antrodia camphorates, and anticancer agents or compositions comprising thereof, have medical effects as described above and have been attracted attention of people widespread, it still cannot be used as a normal anti-tumor agent or used as sole-therapy drug for the treatment of cancer, due to it is still not clear exactly know what is the particular active ingredient or bioactive composition presented therein. As to medicine and pharmacy since, there is need of a drug for treatment or amelioration of cancer with satisfactory effect, and thus it is concerned, in particular, how to fully get appropriate medication and medical efficacy from these extracts of Antrodia camphorates.

Therefore, there is urgent need to develop a pharmaceutical composition which is capable of solving the defects existed in traditional cancer drugs of prior arts and providing excellent medical effects of treating cancer, particularly a combination of different mechanisms of cancer therapeutics.

### SUMMARY OF THE INVENTION

In view of defects and problems as above-mentioned, the inventors of the present application conduct several researches with respect to those problems remained in conventional technologies of prior arts. As results, while an pharmaceutical composition or combination comprising active ingredients or components such as sesquiterpenoids, a eiterpenoids or triterpenoids obtained from the extraction Antrodia camphorates, is used in the treatment and/or prevention of cancers, it is surprising to find that unexpected excellent effects are achieved as compared to the effects offered by traditional anti-cancer agent used in prior arts. Those unexpected excellent effects at least includes for examples, reducing or regulating carcinogenic activity, preventing proliferation or even reversing of cancer cells, and also treating and/or preventing cancer and tumor metastasis.

In addition, the pharmaceutical composition or combination formed by the active ingredients or components the same as that mentioned above, it also found not only having excellent characteristics chemistry, biology, mechanical science and physical science, but also having good performance of transmittance and transportation. It is very easy for the user uptake, in short digestion and absorption, and can be used as drugs or adjuvant for the treatment and/or prevention of cancers, especially can inhibit the prevention and treatment of cancer with efficacy while applied to specific cancer cells. Thus, the present invention is achieved.

Namely, according to one aspect of the invention, a pharmaceutical composition for treating cancer in an individual subject suffering cancer in need such a treatment, comprising at least a pharmaceutically effective amount of a novel target HCC reversal signal substance, and a pharmaceutically acceptable ingredient comprising a vehicle, a carrier, a diluent or an excipient; in which the ratio among each component of the pharmaceutical composition may be adjustable depending on the type of cancer; wherein the novel target HCC reversal signal substance comprises at least one selected from the group consisting of antroquinonol (AQL), antrocinnamonin A (ACA), antroquinonol B (AQB), antroquinonol D (AQD), dehydroeburicoic acid (DEA), dehydrosulphurenic acid (DSA), zhankuic acid A (ZAA), zhankuic acid C (ZAC), antcin K (ANK), antcin C (ANC), and a mixture thereof.

Further, according to another aspect of the invention, the pharmaceutical composition as described above, wherein with respect to the total amount of the triterpenoids presented in the composition, the total amount of antroquinonol (AQL), antrocinnamonin A (ACA), antroquinonol B (AQB) and antroquinonol D (AQD) is in a range of about 0.01 wt. % to about 65.0 wt. %.

Furthermore, according to another one aspect of the invention, the pharmaceutical composition as described above, wherein with respect to the total amount of the triterpenoids presented in the composition, the total amount of dehydroeburicoic acid (DEA) and dehydrosulphurenic acid (DSA) is in a range of about 0.01 wt. % to about 55.0 wt. %.

Besides, according to another one aspect of the invention, the pharmaceutical composition as described above, wherein with respect to the total amount of the triterpenoids presented in the composition, the total amount of zhankuic acid A (ZAA) and zhankuic acid C (ZAC) is in a range of about 0.01 wt. % to about 55.0 wt. %.

In addition, according to another one aspect of the invention, the pharmaceutical composition as described above, wherein with respect to the total amount of the triterpenoids presented in the composition, the total amount of antcin K (ANK) and antcin C (ANC) is in a range of about 0.01 wt. % to about 65.0 wt. %.

Additionally, according to another one aspect of the invention, the pharmaceutical composition as described above, wherein the cancer is liver cancer, colorectal cancer, esophageal cancer, stomach cancer, leukemia, lymphoma, nasopharyngeal carcinoma, brain tumors, lung cancer, breast cancer, cervical cancer, bone cancer, colorectal cancer, liver cancer, breast cancer, or leukemia.

Further, according to another one aspect of the invention, the pharmaceutical composition as described above, wherein the excipient comprises an ingredient selected from the group consisting of lactose, sucrose, a mannitol, sorbitol, maize starch, wheat starch, rice starch, potato starch, gelatin and tragacanth.

Additionally, according to another one aspect of the invention, the pharmaceutical composition as described above, wherein further comprises at least one additive selected from the group consisting of absorption accelerators, antioxidants, binders, buffers, coating agents, coloring agents, diluents, disintegrating agents, emulsifiers, extenders, fillers, flavoring agents, humectants, lubricants, perfumes, preservatives, propellants, releasing agents, bactericides, sweeteners, solubilizers, wetting agents, and a mixture thereof.

### DETAILED DESCRIPTION

In order to make the spirit and content of the present invention more completely and easily to be understood, various examples of the present invention, particularly various specific embodiments are described in more detailed hereinafter. However, a skilled person having general knowledge in this technical field pertains to the present invention, shall understand that the present invention is of course not limited to these examples only, and it is possible to achieve the invention by means of taking advantage of other features of function, efficiency or processes which are the same or equal with the present invention.

First, the descriptive instructions or definitions with respect to a term or a description word or phase particularly used in this specification are separately described below.

Unless otherwise defined in this specification, the meaning of technical terms of science and technology used herein, has the same meaning generally understood and/or used by the skilled person having common knowledge in the field pertains to the present invention.

In this document, a term of “prevention (or prophylaxis)” refers to prevention and control means used for in advance, preventing causing any future event including cancer cell growth and expansion, as well as cancer metastasis causing by surgical or diagnostic procedures.

As used herein, a term of “treatment (or treating)” refers to implementing a preventive, curative or palliative disposal for achievement of pharmaceutical and/or physiological effects to an individual subject or a patient having a certain medical condition, symptoms, disease, disorder or an initial condition, in order to partially or completely reduce the severity, delay the occurrence process, and/or inhibit one or more symptoms of the medical condition, abnormal and/or the probability of occurrence of behavior disorders. The aforementioned symptoms, disease, disorder and/or medical conditions may be in situ or metastatic cancer.

As used herein, a term of “effective amount” refers to while a medical drugs for cancer is administered (administering, or administration) directly or indirectly in a certain amount, and an effect of reducing the number of cancer cells, or particu then lar purpose of treating or preventing a cancer is shown. The said “certain amount” is so called effective amount.

As used herein, the foregoing of “medical drugs” means it is a pharmaceutically active substance capable of inducing expected pharmaceutical and/or physiological effects through local and/or systemic reaction. It generally comprises pharmaceutical compound, formulation, a composition, an agent, pharmaceuticals (medicine or medicament), or a prodrug, a pharmaceutically active compound, a derivative, or analog and others.

As used herein, “individual (subject)” or “patient (patient)” can be used interchangeably with one another. The “individual (or individual subject)” or “patient” means an animal, that can accept a compound and/or method for treatment, for example, including but not limited to, comprise a dog, a cat, a horse, a sheep, a pig, a cattle and any of other mammals, as well as humans, non-human primates. Except as otherwise specifically stated that the “individual (or individual subject)” or “patient” may comprise males and females of both sexes. Also, a preferable individual or patient for treatment by using a pharmaceutical composition and/or a method of the present invention is preferably a human.

In this context, the value of the parameter used for defining the scope of the present invention, in essence, inevitably contain standard deviation caused due to individual test methods, and thus it is mostly expressed by an approximate value of number. However, in particular the implementation of Examples, the value is presented as precisely as possible. In this document, “approximate (or about)” is determined by the skilled person having the usual knowledge of the present invention generally pertains to. Generally, it refers to the actual value which falls in the range of an acceptable standard deviation, for example, the actual value is expressed by a ±10%, it means within a range, ±5%, ±1%, or ±0.5% of a particular value.

The present disclosure is at least partly based on a novel combination of triterpenoods isolated from fruiting body or mycelium of Antrodia camphorates, which is characterized in having capability of anti-proliferation of cancer cells (including a drug-resistant cancer cells). Thus, the novel combination of triterpenoids is a potential agent, which can be used in treatment or prevention of cancer or used as an adjuvant cancer drugs.

Thus, according to the first embodiment of the invention, a pharmaceutical composition for treating cancer in an individual subject suffering cancer in need such a treatment is provided. It comprises at least a pharmaceutically effective amount of antroquinonol (AQL), antrocinnamonin A (ACA), antroquinonol B (AQB), antroquinonol D (AQD), dehydroeburicoic acid (DEA), dehydrosulphurenic acid (DSA), zhankuic acid A (ZAA), zhankuic acid C (ZAC), antcin K (ANK), antcin C (ANC), and a pharmaceutically acceptable ingredient comprising a vehicle, a carrier, a diluent or an excipient; in which the ratio among each component of the pharmaceutical composition may be adjustable depending on the type of cancer. The source for acquiring an active ingredient of pharmaceutical compositions of the present invention is not particularly limited. It can be obtained usually through extracting from mushrooms which is used as a food in common and generally has medicine effect; however it usually is an active ingredient derived from Chi fungus genus by extraction and separation.

Preferably are used those obtained by extraction and separation derived from camphor raw thin volvatus, stout camphor mushroom, Zhang Chi, Zhang Zhi red blood fungus, mushroom inside camphor, or red camphor mushroom, which in taxonomy belongs to Fungi, Basidiomycota, Basidiomycotina, Homobasidiomycetes, Aphyllophorales, Polyporaceae, Antrodia (Antrodia camphorates), and others. More preferably uses an active ingredients derived from an unique Antrodia genus of fungi (Antrodia camphorates, or Antrodia camphorata, Taiwanofungus camphorates) particularly output from Taiwan's mountain through extraction and separation.

Study results of phytochemical researches show that pharmaceutically active ingredient contained in Antrodia camphorata mainly comprises Sesquiterpenoids, Diterpenoids, Triterpenoids, Steroids, furan ring structure such as five member (Furan) or pyrazolyl class (Pyrrole), lignan compound (Lignoids), benzene compounds (Benzenoids), superoxide dismutase and amino acids and the like. The compounds that can be used as pharmaceutically active ingredient of the pharmaceutical composition of the invention, at least comprise AQL, ACA, AQB, AQD, DEA, DSA, ZAA, ZAC, ANK, ANC and another ingredients, of which most are presented in fruiting bodies or mycelium of Antrodia camphorata, and can be obtained by a method of extraction or others.

Specifically, Sesquiterpenoids (sesquiterpene compounds) generally comprise, for example Antrocin and the like.

Diterpenoids (diterpene compounds) generally comprise 19-hydroxylabda-8(17)-en-16,15-olide, 3β,19-dihydroxylabda-8(17),11E-dien-16,15-olide, 13-epi-3β,19-dihydroxylabda-8(17),11E-dien-16,15-olide, 19-hydroxylabda-8(17),13-dien-16,15-olide, 14-deoxy-11,12-didehydroandrographolide, 14-deoxyandrographolide, pinusolidic acid and so on.

Triterpenoids generally comprise Camphoratin B, camphoratin A, Antcin K, Antcin I (zhankuic acid B, 3α-hydroxy-4α-methylergost-8,24(28)-dien-7,11-dione-26-oic acid), camphoratin E, antcin H (zhankuicacid C, 3α,12α-dihydroxy-4α-methylergost-8,24(28)-dien-7,11-dione-26-oic acid), methyl antcinate H (3α,12α-dihydroxy-7,11-dioxo-4α-methylergost-8,24(28)-dien-26-oate), zhankuic acid E, camphoratin C, camphoratin H, camphoratin I, antcin A (1,4α-methylergost-8,4(8)-diene-3,11-dion-26-oic acid), camphoratin J, methyl antcinate A (methyl 4α-methylergost-8,24(28)-dien-3,11-dion-26-oate), antcin E (3,11-dioxo-4α-methylergost-8,14,24(28)-trien-26-oic acid), antcin C (7β-hydroxy-4α-methylergost-8,24(28)-diene-3,11-dion-26-oic acid), camphoratin G, antcin F (3,11-dioxo-7β-hydroxy-4α-methylergost-8,14,24(28)-trien-26-oic acid), camphoratin D, camphoratin F, methyl antcinate G (7α-acetoxy-3,11-dioxo-4α-methylergost-8,24(28)-dien-26-oate), antcin B (zhankuicacid A, 4α-methylergost-8,24(28)-dien-3,7,11-trion-26-oic acid), antcin D (zhankuicacid F, 14-hydroxy-4α-methyl-3,7,11-trioxoergost-8,24(28)-dien-26-oic acid), methyl antcinate B (methyl 4α-methylergost-8,24(28)-dien-3,7,11-trion-26-oate), zhankuic acid D, eburicol (24-methylenedihydrolanosterol), eburicoic acid (35),7 sulphurenic acid, versisponic acid D, dehydroeburicoic acid, dehydrosulphurenic acid, 15α-acetyldehydrosulphurenic acid, 3β,15α-dihydroxylanosta-7,9(11),24-triene-21 oicacid, epi-friedelinol and so on.

Steroids generally comprise β-Sitosterol, stigmasterol (44),16 ergosterol peroxide, ergosterol D, ergosterol, β-sitostenone, ergosta-4,7,8(14),22-tetraen-3-one, ergosta-2,4,8(14),22-tetraen-3-one an so on.

Furan ring structures such as five (Furan) class or pyrazolyl (Pyrrole) class generally comprise Antrocinnamomin C (3-isobutyl-4-(4-hydroxyphenyl)furan-2,5-dione), 3-isobutyl-4-[4-(3-methyl-2-butenyloxy)phenyl]furan-2,5-dione, antrocinnamomin D (2-hydroxy-3-isobutyl-4-[4-(3-methylbut-enyloxy)phenyl]-2H-furan-5-one), cis-3-(4-hydroxyphenyl)-4-isobutyl-dihydrofuran-2,5-dione, dimethyl 2-(4-hydroxyphenyl)-3-isobutyl-maleate, 3-(4-hydroxyphenyl)-4-isobutyl-1H-pyrrole-2,5-dione, 3-iso-utyl-4-[4-(3-methyl-2-butenyloxy) phenyl]-1Hpyrrole-2,5-dione (antrodin B, camphorataimide B), trans-3-isobutyl-4-[4-(3-methyl-2-butenyloxy)phenyl]pyrrolidine-2,5-dione, antrocinnamomin B (3-isobutyl-4-(4-hydroxyphenyl)-1H-pyrrol-1-ole-2,5-dione), 3-isobutyl-4-[4-(3-methyl-2-butenyloxy)phenyl]-1H-pyrrol-1-ol-2,5-dione (antrodin C, camphorataimide C), antrocinnamomin A (3-isobutyl-4-[4-(3-methyl-2-butenyloxy)phenyl]-1H-pyrrol-1-acetoxyl-2,5-dione), trans-1-hydroxy-3-(4-hydroxyphenyl)-4-isobutylpyrrolidine-2,5-dione, 3R,4S-1-hydroxy-3-isobutyl-4-[4-(3-methyl-2-butenyloxy) phenyl]pyrrolidine-2,5-dione), antrodin D (camphorataimide D, 3R, 4R-1-hydroxy-3-isobutyl-4-[4-(3-methyl-2-butenyloxy)phenyl]pyrrolidine-2,5-dione), antrodioxolanone and so on.

Lignoids generally comprise (+)-sesamin, (−)-sesamin, 4-hydroxysesamin, Aptosimon and so on.

Benzenoids generally comprise 1,4-dimethoxy-2,3-methylene-dioxy-5-methylbenzene, methyl 2,5-di-ethoxy-3,4-methylene-dioxybenzoate, 4,5-dimethoxy-2,3-methylene-dioxybenzoicacid, 2,4,5-trimethoxybenzaldehyde, 2,3-methylene-dioxy-6-methylbenzene-1,4-diol, 2,4-dimethoxy-6-methylbenzene-1,3-diol, benzocamphorin C, 5-methylbenzo[1,3]-dioxole-4,7-dione, 2-methoxy-5-methyl[1,4]benzo-quinone, 2,3-dimethoxy-5-methyl[1,4]benzoquinone, isobutylphenol, 2,3,4,5-tetramethoxybenzoylchloride, 2,2,5,5,-tetramethoxy-3,4,3,4,-bis(methylenedioxy)-6,6,-dimethylbiphenyl, benzocamphorin E, benzocamphorin D, antrocamphin A, antrocamphin B, benzocamphorin A, benzocamphorin B and so on.

The other compounds generally comprise α-Tocospiro B, methyloleate, antroquinonol, antroquinonol B, 4-acetylantroquinonol B, adenosine, cordycepin and so on.

The method used for extraction and separation of pharmaceutically active ingredient in the pharmaceutical composition of the present invention, is not particularly limited, for example, can be isolated from the fruiting body or mycelium of Antrodia camphorates by using a conventional purification method well known in prior arts. Whether raw materials are the fruiting body or mycelium of Antrodia camphorates or not, an extraction method suitable for use in general, includes non-polar solvent extraction, highly polar solvent extraction, low polar solvent extraction, high temperature extraction, lower temperature extraction method, the combination of their supercritical extraction method or the like. For example, a solvent suitable for used in the present invention typically includes water, alcohol and the like. Further, extraction temperature suitably used in the present invention is generally not particularly limited, for example, it can be conducted at below 0° C., further it also may be conducted in the lower temperature range of 0° C. to 40° C., or at a higher temperature range of above 50° C. to 150° C.

Further, after the processing of above-described extraction, it may further implement another process for example, concentration, separation, purification, or a combination of methods thereof. Furthermore, it also may be after the extraction in higher than room temperature, and then the obtained extract was subjected to a column chromatography. For example, the separation/purification methods suitable for used in the present invention generally comprise a high efficiency liquid chromatography (HPLC), reverse-phase liquid chromatography and others. Also, the extracts of Antrodia camphorates can then further dried until a dry powder of active compound of Antrodia camphorates is obtained.

Besides, according to one aspect of the invention, each component of pharmaceutically active compounds such as antroquinonol (AQL), antrocinnamonin A (ACA), antroquinonol B (AQB), and/or antroquinonol D (AQD), it is unnecessary to exist in the pharmaceutical compositions in together at the same time. In general, the pharmaceutically active compound may be formed from at least one selected from the group consisting of AQL, ACA, AQB, AQD and a mixture thereof. In some specific embodiments, the pharmaceutically active compound of the invention can be formed from only one component, a combination of two ingredients, a combination of three components, or a combination of the four components selected form the group of AQL, ACA, AQB, and AQD.

For example, in the case of using the three components selected by the AQL, ACA, AQB, AQD as pharmaceutically active compound, for example, the pharmaceutically active compound may be a combination of AQL+AQB+AQD, a combination of AQL+ACA+AQD, a combination of AQL+ACA+AQB, or a combination of ACA+AQB+AQD; in the case of using two compositions; the pharmaceutically active compounds may be a combination of AQL+ACA, a combination of AQB+AQD, a combination of AQL+AQD, or a combination of ACA+AQB; and in the use of a single component, for example, pharmaceutically active compound can be the one selected from AQL, ACA, AQB and AQD.

In some preferred embodiments of the invention, the total amount of AQL, ACA, AQB and AQD in summation presented in the pharmaceutical composition is generally in the range of from about 0.01 weight % to 65.0 weight %, with respect to the total amount of the pharmaceutical active compounds presented in the pharmaceutical composition in terms of 100 parts by weight.

In additionally, according to another aspect of the invention, each component of pharmaceutically active compounds such as dehydroeburicoic acid (DEA), and/or dehydrosulphurenic acid (DSA), it is unnecessary to exist in the pharmaceutical compositions in together at the same time. In general, the pharmaceutically active compound may be formed from at least one selected from the group consisting of DEA, DSA, and a mixture thereof. For example, in some specific embodiments, the pharmaceutically active compound of the invention can be formed from only DEA; in another case, the pharmaceutically active compound of the invention can be formed from only DSA.

In some preferred embodiments of the invention, the total amount of DEA and DSA in summation presented in the pharmaceutical composition is generally in the range of from about 0.01 weight % to 55.0 weight %, with respect to the total amount of the pharmaceutical active compounds presented in the pharmaceutical composition in terms of 100 parts by weight.

Further, according to another aspect of the invention, each component of pharmaceutically active compounds such as zhankuic acid A (ZAA) and/or zhankuic acid C (ZAC), it is unnecessary to exist in the pharmaceutical compositions in together at the same time. In general, the pharmaceutically active compound may be formed from at least one selected from the group consisting of ZAA, ZAC, and a mixture thereof. For example, in some specific embodiments, the pharmaceutically active compound of the invention can be formed from only ZAA; in another case, the pharmaceutically active compound of the invention can be formed from only ZAC.

In some preferred embodiments of the invention, the total amount of ZAA and ZAC in summation presented in the pharmaceutical composition is generally in the range of from about 0.01 weight % to 55.0 weight %, with respect to the total amount of the pharmaceutical active compounds presented in the pharmaceutical composition in terms of 100 parts by weight.

Furthermore, according to another aspect of the invention, each component of pharmaceutically active compounds such as antcin K (ANK) and/or antcin C (ANC), it is unnecessary to exist in the pharmaceutical compositions in together at the same time. In general, the pharmaceutically active compound may be formed from at least one selected from the group consisting of ANK, ANC, and a mixture thereof. For example, in some specific embodiments, the pharmaceutically active compound of the invention can be formed from only ANK; in another case, the pharmaceutically active compound of the invention can be formed from only ANC.

In some preferred embodiments of the invention, the total amount of ANK and ANC in summation presented in the pharmaceutical composition is generally in the range of from about 0.01 weight % to 65.0 weight %, with respect to the total amount of the pharmaceutical active compounds presented in the pharmaceutical composition in terms of 100 parts by weight.

When the pharmaceutically active compounds such as AQL, ACA, AQB, AQD, DEA, DSA, ZAA, ZAC, ANK, and/or ANC of the present invention are used in a pharmaceutical composition or a medical drug, the total amount of the pharmaceutically active compounds (i.e., AQL, ACA, AQB, AQD, DEA, DSA, ZAA, ZAC, ANK, and/or ANC) is not particularly limited, but may generally be in the range of about 0.01 weight % to 85.0 weight %, with respect to the total weight of the pharmaceutical composition or the medical drug.

The pharmaceutical composition of the present invention is suitable for use in the treatment of various cancers, for example but not limited to liver cancer, colorectal cancer, esophageal cancer, stomach cancer, leukemia, lymphoma, nasopharyngeal cancer, brain cancer, lung cancer, breast cancer, cervical cancer, bone cancer, colorectal cancer, liver cancer, breast cancer, or leukemia. Preferred are suitable for use in the treatment of liver cancer, colorectal cancer, esophageal cancer, stomach cancer, leukemia, lymphoma, nasopharyngeal carcinoma, brain tumors, lung cancer, breast cancer, cervical cancer, leukemia or bone cancer.

The pharmaceutical composition disclosed in the present invention can be prepared by any pharmaceutical manufacturing process, provided that it is acceptable in medicine or pharmacy. Further, according to one aspect of the present invention, the pharmaceutical compositions disclosed in the invention may be administered via any suitable way or means of administration. For example, capsules, suspensions or tablets medicine may be administrated by oral administration. Also, instead of parenteral administration, it is possible to be administrated by other processes such as a systemic administration, for example, intramuscular injection, vein injection, subcutaneous injection or intraperitoneal injection.

Additionally, in certain embodiments, the pharmaceutical compositions disclosed in the present invention may also be administered by a process of penetrating or infiltrating skin, for example, applied on local skin; or another ways, for example, by endobronchial, intranasal, oral inhalation or nasal drops instillation; it can be administered within rectum.

In the case of oral administration, the pharmaceutical composition of the present invention may be administered with the excipient or may be administered in the absence of excipients. In addition, pharmaceutical compositions of the present invention can also be the formulations comprising various adjuvants, various disintegrants, particle binders or lubricants so as to form a drug in a solid type, for example, a tablet. Also, in one embodiment, lactose or high molecular weight polyethylene glycols can be used. If required, in order to improve release rate of any further pharmaceutically active ingredient, a coating or covering layer, e.g., enteric coatings can also be used. Furthermore, in some embodiments, the pharmaceutical compositions of the present invention may also be configured to be liposomal structure, or bionic structure or bio-systemic structure, optionally it also can be configured to be a particle encapsulated in a soft or hard gelatin capsules, biodegradable coating or capsules.

Additionally, a term of pharmaceutically acceptable excipient described in the present invention, means the ingredient having biocompatibility compatible with the other components presented in the pharmaceutical preparation, composition or combination. For example, the pharmaceutically acceptable excipient may be an encapsulating material or various additives such as absorption accelerators, antioxidants, binders, buffers, coating agents, coloring agents, diluents, disintegrating agents, emulsifiers, extenders, fillers, flavoring agents, humectants, lubricating a variety of agents, perfumes, preservatives, propellants, releasing agents, bactericides, sweeteners, solubilizers, wetting agents and mixtures thereof.

Adjuvants suitable for use in the present invention, for example, may be microcrystalline cellulose, calcium carbonate, dicalcium phosphate or glycine. Disintegrating agents suitable for use in the present invention, for example, may be starch, alginic acid or a specific silicate. Binder particles suitable for use in the present invention, for example, may be polyvinyl pyrrolidone, sucrose, gelatin, or acacia. Lubricants suitable for use in the present invention, for example, may be magnesium stearate, sodium lauryl sulfate or talc. Excipients suitable for use in the present invention, for example, may be lactose, sucrose, mannitol, sorbitol, corn starch, wheat starch, rice starch, potato starch, gelatin, or tragacanth.

In some embodiments, the pharmaceutical compositions of the present invention may be formed in a liquid formulation which is suitable for use in oral administration, for example, oral suspensions, emulsions, micro-emulsions, and/or curing liquid (elixirs). In the case of the liquid formulation, the active ingredients of the pharmaceutical compositions of the present invention may be further blended with various formulations, for example, sweetening or flavoring agents, coloring matter or dyes, if necessary, it may be further blended with emulsifying and/or suspending agents, or such as water, alcohol, propylene glycol, glycerin and other diluents, or maintain buffer pH values.

Besides, in other embodiments, the formulations comprising a liquid pharmaceutical composition of the present invention may be prepared into sterile injectable solutions or suspensions; for example, it may be manufactured into a solution that is suitable for intravenous injection, intramuscular injection, it in intraperitoneal injection, or subcutaneous administration, and others.

Diluents suitable for use in a sterile injectable solution or suspension described above, for example, may include, but are not limited to, 1,3-butanediol, mannitol, water, Ringer's solution, isotonic chloride sodium; optionally natural oils or fatty acids acceptable in pharmacy, such as oleic acid, glycerol derivatives, or such as olive oil or canola oil, and the like.

In addition, in certain embodiments, alcohol, carboxymethyl cellulose dispersants, surfactants, emulsifiers, or the like may also be further added.

Additionally, in some embodiments, a pharmaceutical composition of the present invention may also be a liquid formulation and may also be filled in a soft capsule for easy use in administration.

Besides, in other embodiments, the aforesaid pharmaceutical or pharmaceutical composition of the present invention may be formed together with a variety of inertial carrier into a dosage form which is suitable for topical administration by applying onto the skin surface, for example, solutions, emulsions, creams, gels, ointments, sprays, skin patch and the like. Typical inertial carriers, for example, may be water, ethanol, polyvinylpyrrolidone, polyethylene glycol, mineral oil, stearic alcohol, or a substance capable of producing a gel.

Further, in some embodiment, he aforesaid pharmaceutical or pharmaceutical composition of the present invention may be formed together with polymer adjuvants to be made into various application forms, for example, mucosal application form, or buccal and/or sublingual form and others. In general, the above-described polymer adjuvant comprises a hydrophilic polymer, for example, it may include, but are not limited to, an acrylic acid polymer, hydrolyzed polyvinylalcohol, polyethylene oxides, polyacrylates, vinyl polymers, polyvinyl pyrrolidine, glucose, dextran, guar gum, pectins, starch, or ellulosic polymers and the like.

In some embodiments, the pharmaceutical compositions of the present invention can be used as another adjuvant for use in therapy, so as to enhance treatment efficacy of cancer including main treatment methods such as surgery, radiation therapy, or chemotherapy. In the case of administration of the pharmaceutical compositions disclosed in the present invention, it can be administered alone or administered in combination with conventional pharmaceutically acceptable adjuvants. For example, the pharmaceutical compositions of the present invention may be administered to a subject through mere an oral administration, or administration together with the food.

In certain embodiments of a method disclosed in the present invention, in order to improve the treatment efficiency of individual's cancer, it may further comprises additional surgery for the individual, radiation therapy or chemotherapy and the like other the main treatment for cancer, prior to, simultaneously with or after administration of the pharmaceutical compositions of the present invention to the subject.

In the general case of that pharmaceutical compositions of the present invention is administered to a patient or individual, with respect to the weight of the patient or the individual, the effective dose or the total amount is usually in the range of about 0.01 mg/kg to about 100.0 mg/kg. Further, the effective dose or the total amount of the pharmaceutical composition per day may be administered to the subject is usually in the range of about 0.01 mg/kg-day to about 50.0 mg/kg-day, with respect to the weight of the patient or the individual. The total dose or amount referred to herein, may be in a single administration in a day; or optionally divided into multiple administrations in a day.

### EXAMPLE

The contents and scopes of the present invention are illustrated hereinafter for exemplification, by some specific embodiments listed as following, however, the contents of the scope of the present invention is not limited those embodiments merely.

Firstly, in 10 cm culture dishes with Dulbecco's modified Eagle's medium (DMEM), Human breast cancer cell line MCF-7, Human hepatocarcinoma cell lines HepG2, Human colorectal cancer cell line HCT116; RPMI medium to foster Human colon cancer cell lines, respectively, RKO, Human tissue leukocyte cell line U937, and pre-Human leukocyte myeloid cell lines HL-60 were cultured; and then 10% fetal calf serum (FCS), 100 units/ml of penicillin, 100 ng/ml of streptomycin (Invitrogen, Carlsbad, Calif.), 2 mM L-glutamic acid and sodium pyruvate and non-essential amino acids were added into the culture medium, and maintained in 37° C. in wet conditions (5% CO2 and 95% air), and while individual cells grew up till 80% of full fill, the subculture was then proceeded.

In the subculture, old culture medium was firstly removed out by absorbing and then cleaning or washing the cultured cells one time with 3 ml of phosphate buffered saline (PBS). Then, the cells were treated at 37° C., with 1 ml of trypsin/EDTA (0.05%/0.025%) in 5 minutes such that the cells were suspended from the culture dishes into a floating state. In addition, by adding 2 ml of fresh culture medium into the suspension cells to neutralize the trypsin activity. Then, the cells were broken up such that the cells averagely distributed in cell suspension, leaving appropriate number of cells in a desired ratio, the new medium was added up to a suitable volume and after mixing, into cultured at 37° C.

About 3,000 cells were taken and seeded them in 96-well flat-bottomed pan, and a broth is added in the culture broth of each hole to a volume of 180 L. After then, the plates were placed in the incubator for 37° C. overnight. The next day, the pharmaceutically active compounds containing different concentrations of pharmaceutical compositions of the invention were added in the above-mentioned culture medium. Each culture wells to a final volume of 200 μL, placed in 37° C. incubator for 48 hours , and after then cell viability was measured.

The cell activity is calculated on basis of the absorbance of p-nitrophenol (p-NP), which is transformed from p-nitrophenyl phosphate (p-NPP) by a large number of ACP contained in living cells, and the absorbance was measured at 405 nm of the wavelength.

During the determination of cell viability, the culture solution is firstly removed out, and it was washed with 200 μL PBS one time, added 100 μl of an analysis buffer with pH about 5.5 (10 mM p-NPP, 0.1 M sodium acetate, 0.1% Triton X-100) therein. The reaction was proceeded at 37° C. for 30-40 minutes, and then 10 μl of 0.1 N sodium hydroxide was added therein to terminate the reaction. After then, it was measured at a wavelength of 405 nm and the measured absorbance was regarded as the activity value of cells. Thus, the activity value of each of a various cells treated under different conditions can be separately calculated, while the activity value of a cell in the control group without the drug administration to be set as 1.

The active ingredient (that is, AQL, ACA, AQB, AQD, DEA, DSA, ZAA, ZAC, ANK, and/or ANC) separated from an extraction extracted or derived from the mycelium or fruiting bodies of Antrodia camphorates, by using the previously described separation or purification method of the present invention, was uniformly mixed according to the composition ratio shown in table 1 to be formulated as the pharmaceutical compositions of the present invention used for a prevention and/or treatment of a particular cancer.

TABLE 1
The Pharmaceutical Compositions of The Invention

| | Composition A | Composition B | Composition C |
|---|---|---|---|
| Effective active | Amount | Amount | Amount |
| component | (weight %) | (weight %) | (weight %) |
| AQL + ACA + | 25 | 30 | 35 |
| AQB + AQD | | | |
| DEA + DSA | 25 | 30 | 15 |
| ZAA + ZAC | 25 | 30 | 15 |
| ANK + ANC | 25 | 10 | 35 |
| Total amount | 100 | 100 | 100 |
| of Effective | | | |
| active component | | | |

AQL: Antroquinonol
ACA: Antrocinnamonin A
AQB: Antroquinonol B
AQD: Antroquinonol D
DEA: Dehydroeburicoic Acid
DSA: Dehydrosulphurenic Acid
ZAA: Zhankuic Acid A
ZAC: Zhankuic Acid C
ANK: Antcin K
ANC: Antcin C

By using the same manner as the method described above, respectively human breast cancer cell line MCF-7, human hepatoma cell line HepG2, human colorectal cancer cell line HCT116, human colon cancer cell line RKO, human tissue leukocyte cell line U937, and bone marrow leukocyte cell line HL-60 was cultured. After then, the pharmaceutical composition A, pharmaceutical composition B, pharmaceutical composition C of the present invention was applied separately, and then ACP activity for each sample was measured to assess the inhibition effectiveness of various cancer cell activity while using pharmaceutical compositions A, B, C of the present invention.

According to those test results of cancer suppression activity, it confirmed that human breast cancer cell line MCF-7, human hepatoma cell line HepG2, human colorectal cancer cell line HCT116, the activity of human colon cancer cell line RKO, human tissue leukocyte cell line U937, and pre-human myeloid white blood cells HL-60 cell line can be suppressed by the pharmaceutical compositions A, B, C of the present invention, particularly all of IC50 (μg/ml) are significantly better than the prior art. In summary, the results show that the pharmaceutical composition of the present invention is a potentially useful drug having pharmaceutical effectiveness in the treatment of various cancers.

It shall be understood that the embodiments and examples as shown above are merely illustrative, and to which various changes, amendments or modifications can be done by those skilled in the art. The specification, embodiment and information provided above is the purpose to make the formation of the specification complete, and to show the embodiment of the present invention merely. Although the present disclosure has been revealed in the embodiment described above, it is not intended to limit the present disclosure, any person skilled in the art can make a variety of modifications and variations to those, without departing from the spirit and scope of the present disclosure, and therefore the scope of the present disclosures of the application will be defined as claims attached herewith.

All of various literature, documents, technical references, patents including patent applications and non-patent publications cited herein, are entirety incorporated by reference, and should not be construed in any way as to limit the invention creative spirit and scope of the right.

What is claimed is:

1. A pharmaceutical composition for treating cancer in an individual subject suffering cancer in need such a treatment, comprising at least a pharmaceutically effective amount of a novel target HCC reversal signal substance, and a pharmaceutically acceptable ingredient comprising a vehicle, a carrier, a diluent or an excipient; in which the ratio among each component of the pharmaceutical composition may be adjustable depending on the type of cancer;
wherein the novel target HCC reversal signal substance comprises at least one selected from the group consisting of antroquinonol (AQL), antrocinnamonin A (ACA), antroquinonol B (AQB), antroquinonol D (AQD), dehydroeburicoic acid (DEA), dehydrosulphurenic acid (DSA), zhankuic acid A (ZAA), zhankuic acid C (ZAC), antcin K (ANK), antcin C (ANC), and a mixture thereof.

2. The pharmaceutical composition as described in claim 1, wherein with respect to the total amount of the triterpenoids presented in the composition, the total amount of antroquinonol (AQL), antrocinnamonin A (ACA), antroquinonol B (AQB) and antroquinonol D (AQD) is in a range of about 0.01 wt. % to about 65.0 wt. %.

3. The pharmaceutical composition as described in claim 1, wherein with respect to the total amount of the triterpenoids presented in the composition, the total amount of dehydroeburicoic acid (DEA) and dehydrosulphurenic acid (DSA) is in a range of about 0.01 wt. % to about 55.0 wt. %.

4. The pharmaceutical composition as described in claim 1, wherein with respect to the total amount of the triterpenoids presented in the composition, the total amount of zhankuic acid A (ZAA) and zhankuic acid C (ZAC) is in a range of about 0.01 wt. % to about 55.0 wt. %.

5. The pharmaceutical composition as described in claim 1, wherein, with respect to the total amount of the triterpenoids presented in the composition, the total amount of antcin K (ANK) and antcin C (ANC) is in a range of about 0.01 wt. % to about 65.0 wt. %.

6. The pharmaceutical composition as described in claim 1, wherein the cancer is liver cancer, colorectal cancer, esophageal cancer, stomach cancer, leukemia, lymphoma, nasopharyngeal carcinoma, brain tumors, lung cancer, breast cancer, cervical cancer, bone cancer, colorectal cancer, liver cancer, breast cancer, or leukemia.

7. The pharmaceutical composition as described in claim 1, wherein the individual subject is a human.

8. The pharmaceutical composition as described in claim 1, wherein the excipient comprises an ingredient selected from the group consisting of lactose, sucrose, a mannitol, sorbitol, maize starch, wheat starch, rice starch, potato starch, gelatin and tragacanth.

9. The pharmaceutical composition as described in claim 1, wherein further comprises at least one additive selected from the group consisting of absorption accelerators, antioxidants, binders, buffers, coating agents, coloring agents, diluents, disintegrating agents, emulsifiers, extenders, fillers, flavoring agents, humectants, lubricants, perfumes, preservatives, propellants, releasing agents, bactericides, sweeteners, solubilizers, wetting agents, and a mixture thereof.
